(19) United States
(12) Patent Application Publication
(10) Pub. No.: US 2020/0164439 A1
(43) Pub. Date: May 28, 2020

(54) POROUS BIOMEDICAL IMPLANT AND MANUFACTURING METHOD THEREOF

(71) Applicant: METAL INDUSTRIES RESEARCH & DEVELOPMENT CENTRE, Kaohsiung City (TW)

(72) Inventors: Meng-Hsiu Tsai, Kaohsiung City (TW); Tai-I Hsu, Tainan City (TW); Chun-Chieh Wang, Kaohsiung City (TW); Chia-Min Wei, Tainan City (TW)

(21) Appl. No.: 16/200,787

(22) Filed: Nov. 27, 2018

(51) Int. Cl. B22F 3/11; B22F 3/105; B33Y 10/00; B33Y 80/00; A61F 2/30

(52) U.S. Cl. CPC B22F 3/11; B22F 3/1055; B33Y 10/00; B33Y 80/00; A61F 2002/30985; B22F 2003/1058; A61F 2002/30784; A61F 2002/3093; A61F 2/30771

(57) ABSTRACT

A manufacturing method of a porous biomedical implant includes the steps of providing a supporter having a bearing surface, forming the porous biomedical implant on the bearing surface by additive manufacturing and removing the supporter after additive manufacturing. The porous biomedical implant includes a solid part and a porous part, the solid part is coupled to the bearing surface of the supporter and the porous part is coupled to the solid part. Particularly, the solid and porous parts are created in same layers by additive manufacturing.

### FIELD OF THE INVENTION

This invention generally relates to a biomedical implant, and more particularly relates to a porous biomedical implant and manufacturing method thereof.

### BACKGROUND OF THE INVENTION

Currently, implanting biomedical implants into organism to replace original joint, intervertebral disc or tooth root is a well-developed technology, and there are many studies regarding structure or material of various biomedical implants. Taiwan patent application no. 102144752 (patent no. TW 1615136) discloses an intervertebral implant and manufacturing method thereof. A support model made of porous hydroxyapatite is sintered and molded with metal powders filled in the support mounting model to form the intervertebral implant having local degradable hydroxyl apatite/metal block. This prior art utilizes combined material to make a porous intervertebral implant, but the manufacturing method is complex because the steps of degradable material sintering, metal powders filling/sintering and subsequent processing are required.

### SUMMARY

The object of the present invention is to provide a porous biomedical implant manufactured by additive manufacturing. The porous biomedical implant includes a solid part for supporting and a porous part for bone cell growth. The complexity of the manufacturing method of the present invention is reduced significantly due to the solid and porous parts are produced by additive manufacturing and supporter removing is the only step after additive manufacturing.

A manufacturing method of the porous biomedical implant of the present invention includes the steps of providing a supporter having a bearing surface, forming a porous biomedical implant on the bearing surface by additive manufacturing and removing the supporter after additive manufacturing. The porous biomedical implant includes a solid part and a porous part created by additive manufacturing, the solid part is coupled to the bearing surface and the porous part is coupled to the solid part.

The porous biomedical implant of the present invention is manufactured by additive manufacturing. The solid part in the porous biomedical implant is designed to provide enough mechanical support and the porous part in the porous biomedical implant is designed to provide pore spaces for bone cell growth. Furthermore, the complexity of subsequent processes can be lower significantly due to the solid and porous parts are created by additive manufacturing directly.

### DESCRIPTION OF THE DRAWINGS

FIG. 1 is a flow chart illustrating a manufacturing method of porous biomedical implant in accordance with one embodiment of the present invention.

FIG. 2 is a schematic diagram illustrating a supporter in accordance with one embodiment of the present invention.

FIG. 3 is a schematic diagram illustrating a porous biomedical implant formed on the supporter in accordance with one embodiment of the present invention.

FIG. 4 is a cross-section view diagram illustrating the porous biomedical implant in accordance with one embodiment of the present invention.

FIG. 5 is a schematic diagram illustrating the porous biomedical implant without the supporter in accordance with one embodiment of the present invention.

### DETAILED DESCRIPTION OF THE INVENTION

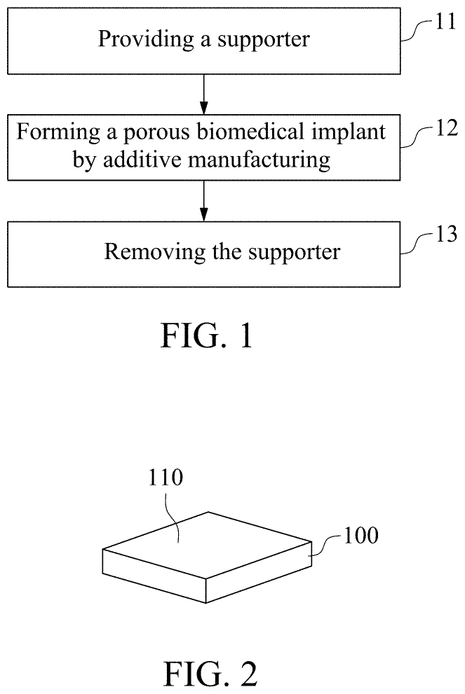

With reference to FIG. 1, a manufacturing method 10 of a porous biomedical implant in one embodiment includes step 11 of providing a supporter, step 12 of forming a porous biomedical implant by additive manufacturing and step 13 of removing the supporter. In this embodiment, electron beam is provided as a heat source for additive manufacturing in the steps 11 and 12. However, the heat source of additive manufacturing may be laser or plasma in other embodiments. Electron beam additive manufacturing (EBAM) is a well-known 3D printing technology so the details of conventional processes, such as modeling, vacuum pumping, powder feeding, air venting and powder recycling, are not repeated here.

With reference to FIGS. 1 and 2, a supporter 100 is provided in the step 11. In this embodiment, the supporter 100 is manufactured on an elevating platform (not shown) by additive manufacturing and is able to move with the elevating platform that is beneficial for layer-by-layer construction during additive manufacturing. The supporter 100 has a bearing surface 110 used to bear biomedical implant formed subsequently.

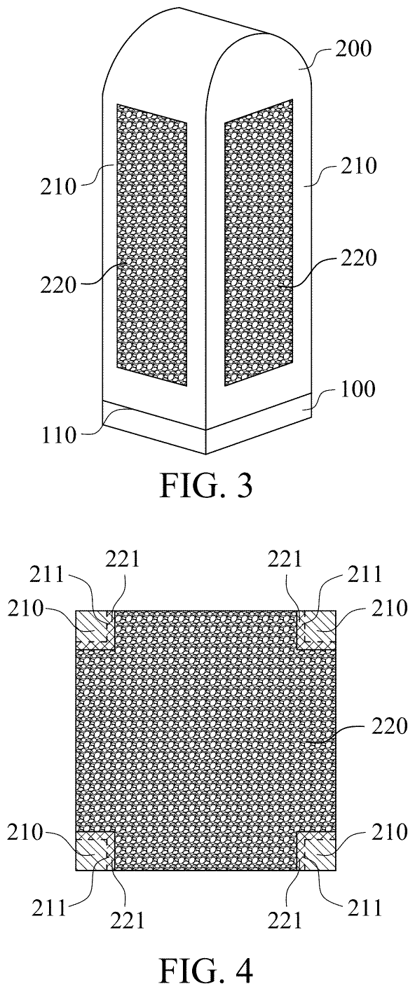

With reference to FIGS. 1 and 3, a porous biomedical implant 200 is formed on the bearing surface 110 of the supporter 100 by additive manufacturing in the step 12. The porous biomedical implant 200 includes a solid part 210 and a porous part 220, the solid part 210 has a higher sinter density so that it can provide the needed mechanical support for the porous biomedical implant 200 during compression, torsion or fatigue. The solid part 210 is formed on the bearing surface 110 of the supporter 100. There are many pores in the porous part 220 provided for bone cells growth after implanting, and preferably, the pores are through-pores. Owing to the porous part 220 has a lower sinter density, it does not need the supporting of the supporter 100 and is formed on and connected to the solid part 210. In this embodiment, the porous biomedical implant 200 is a lumbar cage, accordingly, sufficient supporting capacity and pores for self-growth are necessary.

FIG. 4 schematically illustrates a cross-section view of the porous biomedical implant 200 viewed in the additive direction. The solid part 210 and the porous part 220 are formed in same layers of the porous biomedical implant 200 along the additive direction and they have different sinter densities. Even though the solid part 210 and the porous part 220 are formed in same layers during additive manufacturing, a first manufacturing parameter for the solid part 210 and a second manufacturing parameter for the porous part 220 are different such that the solid part 210 having good supporting effect and the porous part 220 having pores can be formed in the same layers of the porous biomedical implant 200.

In this embodiment, the first and second manufacturing parameters for forming the solid part 210 and the porous part 220 are the energy densities. The energy density of the first manufacturing parameter is preferably between 0.15 J/mm and 0.30 J/mm and the energy density of the second manufacturing parameter is preferably between 0.10 J/mm and 0.12 J/mm, as a result, the solid part 210 with adequate mechanical property and the porous part 220 designed for bone cells growth can be formed in the same layers during additive manufacturing.

The energy densities of the first and second manufacturing parameters are controlled by the voltage and the current of the electron beam and the scanning speed of the heat source in this embodiment. In the first manufacturing parameter, the voltage is 60000 V, the current is between 12 mA and 20 mA and the scanning speed of the heat source is between 4000 mm/s and 10000 mm/s. Different to the first manufacturing parameter, the voltage is 60000 V the current is from 3 mA to 5 mA and the scanning speed of the heat source is from 1000 mm/s to 3000 mm/s in the second manufacturing parameter.

With reference to FIG. 4, the porous part 220 is preferably formed in the same layer after forming the solid part 210 during additive manufacturing. Moreover, in the same layer, the porous part 220 within a porous overlapping region 221 overlaps the solid part 210 within a solid overlapping region 211. In other words, the porous part 220 in the porous overlapping region 221 is designed to overlap the solid part 210 in the solid overlapping region 211 during modeling such that the heat source will scan the solid part 210 in the solid overlapping region 211 again when forming the porous part 220. The solid part 210 in the solid overlapping region 211 is not influenced by the secondary heat process and is able to be connected to the porous part 220 steadily.

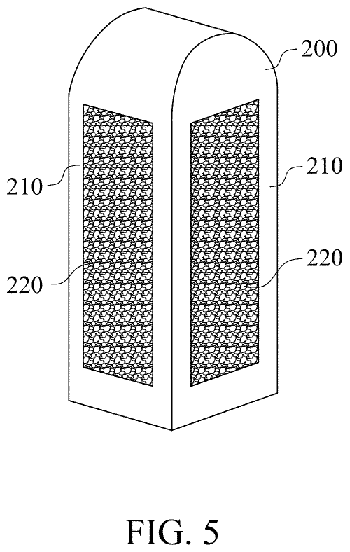

With reference to FIGS. 1 and 5, owing to the external and internal forms of the solid part 210 and the porous part 220 are manufactured by additive manufacturing directly, the manufacturer only needs to remove the supporter 100 after additive manufacturing to obtain the porous biomedical implant 200.

The porous biomedical implant 200 of the present invention has the solid part 210 and the porous part 220 manufactured by additive manufacturing, the solid part 210 is designed to provide sufficient mechanical support and the porous part 220 is designed to hold the bone cells for cell growth. Moreover, the complexity of the manufacturing method can be reduced dramatically because the solid part 210 and the porous part 220 are manufactured by additive manufacturing directly.

While this invention has been particularly illustrated and described in detail with respect to the preferred embodiments thereof it will be clearly understood by those skilled in the art that is not limited to the specific features shown and described and various modified and changed in form and details may be made without departing from the spirit and scope of this invention.

What is claimed is:

1. A manufacturing method of porous biomedical implant, comprising:
providing a supporter having a bearing surface;
forming a porous biomedical implant by an additive manufacturing process, the porous biomedical implant includes a solid part and a porous part, the solid part is coupled to the bearing surface of the supporter and the porous part is coupled to the solid part, wherein the solid part and the porous part are formed in same layers of the porous biomedical implant during the additive manufacturing process; and
removing the supporter after the additive manufacturing process of the porous biomedical implant.

2. The manufacturing method of porous biomedical implant in accordance with claim 1, wherein the solid part and the porous part are formed by a first manufacturing parameter and a second manufacturing parameter respectively in the additive manufacturing process of the porous biomedical implant, and the first manufacturing parameter is different to the second manufacturing parameter.

3. The manufacturing method of porous biomedical implant in accordance with claim 2, wherein the first and second manufacturing parameters are two energy densities respectively.

4. The manufacturing method of porous biomedical implant in accordance with claim 3, wherein the energy density of the first manufacturing parameter is between 0.15 J/mm and 0.30 J/mm and the energy density of the second manufacturing parameter is between 0.10 J/mm and 0.12 J/mm.

5. The manufacturing method of porous biomedical implant in accordance with claim 4, wherein a heat source used in the additive manufacturing process is an electron beam.

6. The manufacturing method of porous biomedical implant in accordance with claim 5, wherein the first and second manufacturing parameters both involve a voltage, a current and a scanning speed of the heat source.

7. The manufacturing method of porous biomedical implant in accordance with claim 6, wherein the voltage is 60000 V, the current is between 12 mA and 20 mA and the scanning speed of the heat source is between 4000 mm/s and 10000 mm/s in the first manufacturing parameter, and the voltage is 60000 V, the current is between 3 mA and 5 mA and the scanning speed of the heat source is between 1000 mm/s and 3000 mm/s in the second manufacturing parameter.

8. The manufacturing method of porous biomedical implant in accordance with claim 1, wherein the porous part is formed in the same layer after forming the solid part.

9. The manufacturing method of porous biomedical implant in accordance with claim 8, wherein the porous part in a porous overlapping region overlaps the solid part in a solid overlapping region when the porous part is formed in the same layer after forming the solid part.

10. A porous biomedical implant, comprising:
a solid part formed by an additive manufacturing process according to a first manufacturing parameter; and
a porous part formed by the additive manufacturing process according to a second manufacturing parameter, the porous part is coupled to the solid part, wherein the solid part and the porous part are formed along an additive direction of the porous biomedical implant, and the first manufacturing parameter for the solid part is different to the second manufacturing parameter for the porous part.

11. The porous biomedical implant in accordance with claim 10, wherein the porous part is formed in same layers after forming the solid part.

12. The porous biomedical implant in accordance with claim 10, wherein the porous part in a porous overlapping region overlaps the solid part in a solid overlapping region.

13. The porous biomedical implant in accordance with claim 11, wherein the porous part in a porous overlapping region overlaps the solid part in a solid overlapping region.
